(19) United States
(12) Patent Application Publication
(10) Pub. No.: US 2006/0161094 A1
(43) Pub. Date: Jul. 20, 2006

(54) 3D clampable valve flow reversing system

(76) Inventors: David S. Utterberg, Seattle, WA (US); David Bell, Grayslake, IL (US)

Correspondence Address: Garrettson Ellis;Seyfarth Shaw LLP, Suite 4200, 55 East Monroe Street, Chicago, IL, 60603, US

(21) Appl. No.: 11/036,266

(22) Filed: Jan. 14, 2005

(51) Int. Cl. A61M 37/00

(52) U.S. Cl. 604/6.16

(57) ABSTRACT

A flow reversal valve for a tubular fluid flow set comprises at least four tubular branches and a tubular loop. The tubular loop separately connects with each of the tubular branches at ends thereof, the tubular loop having collapsible, clampable tube sections between connections with the branches. At least some of the tubular branches connect at a transverse, third dimensional angle to the tubular loop, either directly, or through a substantially rigid, angular bend in the blood flow tubing adjacent to the tubular loop.

### BACKGROUND OF THE INVENTION

In Bell et al. U.S. Pat. No. 6,695,807, a blood flow reversing system is disclosed for use in extracorporeal blood treatment, for circulating blood between a patient and an extracorporeal blood treatment device. The set portion has an arterial tube for conveying blood from the patient toward the blood treatment device, and a venous tube for conveying blood from the blood treatment device back towards the patient. A central, typically square, flow reversing section is provided, comprising portions of the arterial and venous tubes, and a pair of spaced, transverse tubes that each connect between the arterial tube and the venous tube. The arterial and venous tube portions and the spaced transverse first tubes are each capable of clamp sealing.

Thus, a blood flow reversing set is provided in which the portions of the arterial and venous tube which are on the patient side of the flow reversing section can have the blood flow reversed, if desired, while the blood flow of the arterial and venous tubes on the extracorporeal device side of the set proceeds in unchanged direction. This has significant advantages which are known to the prior art, and discussed in the above-cited Bell et al. patent, with the flow reversal being accomplished without a rotary valve, as is done in other prior art such as Schnell et al. U.S. Pat. Nos. 6,177,049 and 6,319,465. In Bell, et al. U.S. Pat. No. 6,695,807, simple sliding clamps are used to effect such flow control.

While the system of the Bell et al. patent has significant advantages, there are certain improvements that would be desired in some circumstances. First, in its flat, substantially two-dimensional form as specifically disclosed in the Bell et al. patent, the blood flow reversing system requires that tubings connected thereto have to cross each other, so that the respective arterial and venous tubes that extend to the patient point in the same direction, while the arterial and venous tubes that extend to the extracorporeal blood processing device point in the opposite direction. This can be readily done, but it can add a complexity in the setup. Also, it is of course desirable to reduce the amount of tubing required in an extracorporeal flow system.

By this invention, a blood flow reversing system is provided which does permit the use of a lesser length of tubing overall, all other things being equal, and the device of this invention also has the advantage that the crossing tubings which add complexity can be avoided. Thus, a blood flow reversing set can be provided by this invention which is less expensive, because of the reduction in amount of tubing used, and which is more easily capable of being properly set up.

### DESCRIPTION OF THE INVENTION

In accordance with one aspect of this invention, a flow reversal valve for a tubular fluid flow set comprises at least four tubular branches and a tubular loop, the tubular loop separately connecting with each of the tubular branches at ends thereof. Typically, the tubular loop substantially occupies a plane, and has clampable, collapsible tube sections between the branches (i.e., between the connection sites of the loop and the branches).

In some embodiments, each branch connects with the tubular loop in a direction which occupies a plane with the loop. However, in this case at least some, and typically all, of the tubular branches will also define a substantially rigid, angular bend directing the tubular branch out of the plane.

It is generally preferred for the tubular loop to be essentially quadrilateral in shape, and typically the tubular loop may be of essentially square shape. However, it would be possible to practice this invention with the tubular loop comprising a circle or oval, and other shapes of a closed loop might be used if desired.

As stated, each of the tubular branches can define the angular bend out of the plane described above. In some embodiments, the tubular branches connect to the tubular loop at corners of the square shape. Typically, the angular bends of a pair of the branches at opposite corners of the square shape extend in a first direction which is transverse to the plane of the tubular loop. The angular bends of the remaining branches extend in a transverse direction which is opposite to the first direction.

As used herein, the term “substantially rigid” does not necessarily imply the rigidity of a metal fixture, although metal fixtures can be used and would be broadly included in this invention. The term “substantially rigid” also includes materials having the rigidity of plastic components which are self-supporting and more rigid than flexible, plastic tubing, so that the substantially rigid, angular bends referred to positively direct the tubing of the bends out of the plane of the tubular loop.

In another aspect of this invention, a flow reversal valve for a tubular fluid flow set may comprise at least four tubular branches and a tubular loop, as before, with the tubular loop separately connecting with each of the tubular branches at ends of the branches. The tubular loop may have collapsible, clampable tube sections between the connections with the branches. At least some of the tubular branches connect with the tubular loop at a transverse angle to the tube sections of the tubular loop, to typically direct the tubular branches out of the plane of the tubular loop.

While the tubular loop may be planar, it is not necessarily planar, and could be V-shaped in a side view, for example. However, the tubular loop may occupy a plane, and, typically, may be of essentially rectangular shape. The tubular branches connect directly and transversely to the tubular loop, and they may be straight.

In this second aspect of the invention, some embodiments of the tubular branches may connect to the tubular loop at corners of the square shape, with the direction of connection of branches at opposite corners extending transversely out of the plane of the loop in a first direction, and the direction of connection of the remaining branches extending transversely out of the plane of the loop in a direction opposite to the first direction. In some preferred embodiments, each tubular branch connects transversely with the tubular loop of the loop plane.

Broadly, this invention relates to a flow reversal valve for a tubular flow set comprising at least four tubular branches and a tubular loop. The tubular loop has separate connections with each of the tubular branches at ends of the tubular branches. The tubular loop has clampable (typically collapsible) tube sections between said connections with said branches. The tubular loop and the tubular branches together define a self-supporting structure that defines three dimensionally arranged flow paths through the tubular loop and tubular branches. This arrangement can be used to facilitate the directions in which the tubular branches extend, for efficient operation as described above.

In some embodiments, the tubular loop may comprise a plurality of the tube sections connected together in rectangular form and defining four corners. The tubular branches may connect to the loop at those corners.

In the embodiments described above, a one-piece slide clamp may be provided, capable of collapsing two opposed portions of the tubular loop, to provide selective flow control through the flow reversal valve, depending on the position of the slide clamp. Alternatively, two separate side clamps may be used, if desired.

The flow reversal valve of this invention may be connected to a tubular set either before or after sale to a user, to comprise part of an otherwise conventional, extracorporeal blood handling set or other medical set, in which the portions of the tubular set nearest to the patient can have reversed flow from normal, when that is deemed to be desirable, by manipulation of slide clamps on the tubular loop, while portions of the extracorporeal set which are nearer to the extracorporeal blood treatment apparatus, and the blood pump, may retain the original flow direction through the entire process.

Thus, by this arrangement, the respective tubes extend in more desirable directions than that which is specifically shown in Bell et al. U.S. Pat. No. 6,695,807. Connections may be made with arterial and venous blood flow tubing sections, and they may respectively extend to the patient and to the extracorporeal processing machine, such as a dialyzer, without confusing line crossing, and with the use of a lesser amount of tubing, all other things being equal.

### DESCRIPTION OF THE DRAWINGS

Referring to the drawings, FIG. 1 is a perspective view of a flow reversal valve for a tubular flow set, in accordance with this invention.

FIG. 2 is a plan view of a double acting slide clamp as shown by position lines 2-2, to be in a vertical position for closing two opposed tube sections of the flow reversal valve.

FIG. 3 is a plan view of the slide clamp of FIG. 1 (the position being shown in broken lines in FIG. 1,) with the slide clamp being in a horizontal position as indicated in position line 3-3 for closing the flow reversal valve in a different flow mode from that of FIG. 2.

FIG. 4 is an elevational view of the flow reversal valve system of FIG. 1, connected to other blood flow set components in an extra corporeal blood flow circuit.

FIG. 5 is an enlarged, perspective view of another embodiment of the flow reversal valve for a tubular flow set in accordance with this invention, usable as an alternative to the flow set of FIG. 1.

### DESCRIPTION OF SPECIFIC EMBODIMENTS

Referring to FIGS. 1-4, a flow reversal valve is shown, and particularly shown connected in an extracorporeal flow circuit in FIG. 4.

Flow reversal valve 10 comprises an arrangement of tubular blood flow conduit sections, including a tubular loop 12, shown to be in square configuration, with the tubular loop being connected with four tubular branches 14 which, in turn, are connected to flexible blood flow tubing sections 16, 16a as an extension of the branches 14.

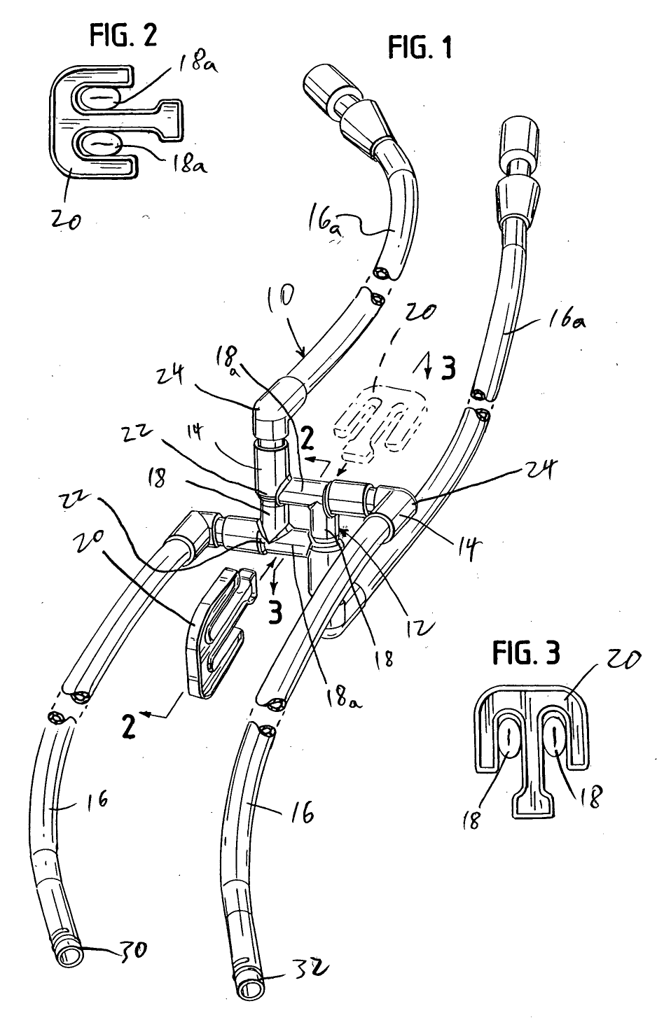

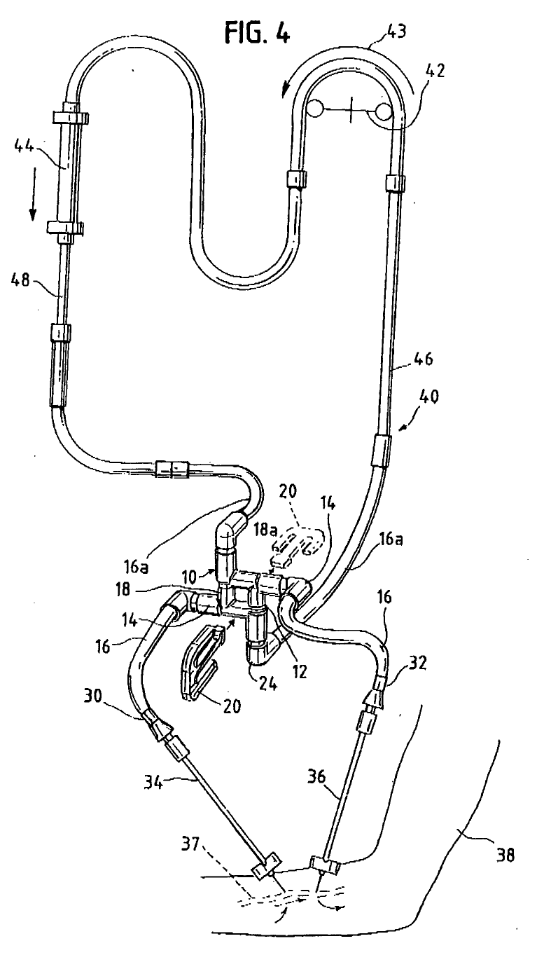

In this embodiment, tubular loop 12 occupies a plane. Also, tubular loop 12 defines clampable, collapsible, flexible tube sections 18, 18a between each connection with each branch 14, so that double acting clamp member 20 can be used to close off a pair of opposite, clampable tube sections 18a that are vertically disposed, by advancement into tubular loop 12 in a vertical position as illustrated in FIG. 1 in full lines and in FIG. 2. In the configuration of FIG. 3, clamp 20 is advanced in a horizontal position, as shown in the broken lines of FIG. 1 and in FIG. 3, to engage the other two opposed, clampable tube sections 18 that are horizontally disposed to each other. Thus, by the use of the single clamp 20, two different flow paths can be defined through flow reversal valve 10, depending upon whether clamp 20 is positioned within tubular loop to clamp an opposed pair of tubular branches that are vertically spaced from each other or horizontally spaced from each other, in a manner of operation that is broadly similar to that of Bell U.S. Pat. No. 6,695,807, which is incorporated herein by reference, except as otherwise described herein.

In accordance with this invention, tubular loop 12 and the respective tubular branches 18, 18a define a self-supporting structure in the immediate vicinity of tubular loop 12 which, in turn, defines three dimensionally arranged flow paths through the tubular loop and the tubular branches. The plastic of tubular branches 14 is sufficiently stiff to positively define the shape shown, although semi-flexible plastic may be used if desired. Each of tubular branches 14 can be seen to communicate with tubular loop 12 at a corner 22 of rectangular loop 12, extending outwardly in a direction that is generally parallel, in this embodiment, to the plane defined by tubular loop 12, and then having an angle 24 that directs the attached tubing 16 into a transverse, third dimension relative to tubular loop 12. By this, a pair of flexible branch tubing portions 16 extend in one direction, in a third, transverse dimension relative to tubular loop 12, being connected to diagonally opposed corners 22 of the tubular loop. The other two flexible, tubular portions 16a of the tubular branches connect through tubular branches 14 to diagonally opposed corners of the tubular loop 12, with tubing 16a extending in the same third, transverse dimension, but in the opposite direction from the direction of extension of tubings 16. By this means, the respective tubings 16, 16a can be more effectively directed in the direction in which they should extend, as particularly shown in FIG. 4, to more efficiently and effectively form an extracorporeal blood flow circuit, with less confusing tube crossing, and with less tubing being required than in some arrangements that utilize the apparatus of U.S. Pat. No. 6,695,807.

As shown in FIG. 4, the flow reversal valve system 10 of this invention is connected through conventional connectors 30, 32 to a respective pair of conventional fistula sets 34, 36, having needles which connect to the vascular system 37 in the arm of a patient 38. Fistula set 34 may normally be the arterial set that withdraws blood from the patient to pass through the connected, tubular branch 16 and into flow reversal valve system 10. Depending on the position of double slide clamp 20, flowing blood is directed through either of the tubular branches 16a into the remainder of a connected, extracorporeal flow set 40, which is shown merely in an exemplary manner, and lacks some of the conventional components and attachments of a conventional extracorporeal flow set, which will normally be present. However, flow set 40 is specifically shown to contain a roller pump system 42 and a hemodialysis or hemoperfusion device 44, which may be of conventional design. In actual use, extracorporeal flow set 40 typically contains branching tubes for fluid connection and pressure sensing, injection sites, safety monitoring devices such as pressure pillows and inserts for optical sensors, drip chambers, and other components that comprise a known, clinically available hemodialysis or other extracorporeal blood flow set. It can be seen that an arterial component 46 and venous component 48 of such conventional flow sets are present.

Returning blood from flow set 40 normally passes again into flow reversal valve system 10, and may be returned by the tubular branch 16 that connects with fistula set 36, for return to the patient.

Thus, as is a known procedure, the flow of blood in extracorporeal set portion 40 is driven in a single flow direction 43 by the action of roller pump 42, which may be a unidirectional flow pump. However, depending upon the position of double clamp 20 as it is inserted into tubular loop 12, vertically or horizontally, a different flow path through tubular branches 16 and their connected fistula sets 34, 36 is provided. When double clamp 20 is inserted to close off horizontally disposed clampable tube sections 18, the flow through the system is from the patient's vein or fistula 37, through fistula set 34 and its connected tube 16, passing through tubular loop 10 to the tubular branch 16a that connects first to pump 42 and then to dialyzer 44. From there, the flow returns again to tubular loop 10, through the tubular branch 16 that connects to fistula set 36, and back to the patient.

However, when clamp 20 is inserted in vertical manner, as illustrated in FIG. 2 and as suggested in the solid line clamp 20 of FIG. 1, to close off vertically disposed clampable tube sections 18a, 18a, roller pump 42 drives flow in reverse through tubular branches 16 and the respective fistula sets, so that blood flow is returned to the patient through fistula set 34, and blood is drawn from the patient through fistula set 36.

As is well known, diagnostic tests may be performed in this reverse flow mode, which is easily achievable to the extracorporeal blood treatment practitioner, simply by manipulation of double acting slide clamp 20 by either placing it vertically or horizontally.

Because of the three-dimensional characteristic of tubular loop, the adjacent portions of tubular sections 14, and their angled corners 24, it is possible for the tubular branch extensions 16, 16a (FIG. 4) to initially extend in opposing directions where crossing of the respective tube extensions 16, 16a is not required for proper connection with fistulas and extracorporeal blood set 40. This simplifies the setup and avoids confusion. Also, since the three-dimensionally angled corners 24 point the tubing extensions in proper directions, the lengths of at least some of the tubing extensions can be shortened, resulting in a net saving of tubing and a consequent cost reduction.

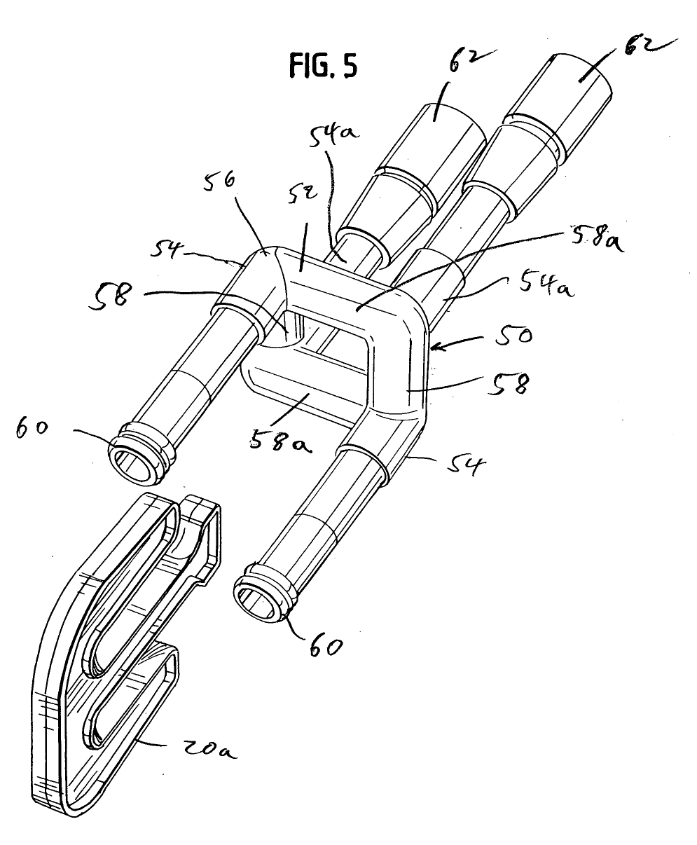

Turning to FIG. 5, another embodiment 50 of a flow reversal valve in accordance with this invention is disclosed. Valve 50 is similar in structure and in function to flow reversal valve 10, comprising a tubular loop 52 which is of rectangular shape, and is connected at the respective four corners with tubular branches 54, 54a in a manner similar to the previous embodiment.

As one distinction, branches 54 connect directly to the respective corners 56 of tubular loop 50 in a manner that is transverse to the tubular loop 50 itself, to provide the desired three-dimensional structure, with the respective tubular branches 54 being diagonally disposed to each other and tubular branches 54a being also diagonally disposed to each other. Double acting slide clamp 20a is provided for a function similar to that of the previous embodiment. When advanced vertically as shown, the vertically spaced or disposed, collapsible sections 58a are closed. If double acting clamp 20a is advanced horizontally, then the horizontally spaced or disposed, collapsible sections 58 are closed, with a consequent controlled, changed in the flow path to reverse flow in the tubular branches 54 that extend to the patient.

In FIG. 5, flow reversal valve 50 is shown as a separate component, carrying female luer connector 60 and male luer lock connector 62 for connection with other components of the system, otherwise operating in a manner similar to that shown in FIG. 4.

Accordingly, a flow reversal valve for a tubular flow set such as a hemodialysis set and system is provided, in which flow between the flow reversal valve and the patient can be reversed, while the flow in the portion of the system which is between the flow reversal valve and the roller pump and extracorporeal blood treatment device remains unchanged. By this invention, simplicity of setup is provided, along with further economy provided by a reduction in length of tubing used, all other things being equal.

The above has been offered for illustrative purposes only, and is not intended to limit the scope of the invention of this application, which is as defined in the claims below.

That which is claimed is:

1. A flow reversal valve for a tubular fluid flow set comprising at least four tubular branches and a tubular loop, said tubular loop having separate connections with each of said tubular branches at ends of said branches, said tubular loop occupying a plane, and having clampable tube sections between said connections with said branches, at least some of said tubular branches connecting with said tubular loop in a direction occupying said plane, but also defining a substantially rigid angular bend, directing a portion of said tubular branch out of said plane.

2. The valve of claim 1 in which said tubular loop is of essentially square shape.

3. The valve of claim 2 in which each tubular branch connects with said tubular loop in a direction occupying said plane and defines said angular bend out of said plane.

4. The valve of claim 3 in which said tubular branches connect to the tubular loop at corners of said square shape, the angular bends of a pair of branches at opposite corners extending in a first direction transverse to said plane, and the angular bends of the remaining branches extending in a transverse direction opposite to said first direction.

5. The valve of claim 1 in which each tubular branch connects with said tubular loop in a direction occupying said plane and defines said angular bend out of said plane.

6. A flow reversal valve for a tubular fluid flow set comprising at least four tubular branches and a tubular loop, said tubular loop having separate connections with each of said tubular branches at ends of said branches, said tubular loop having clampable tube sections between said connections with said branches, at least some of said tubular branches connecting with said tubular loop at a transverse angle to the tube sections of said tubular loop.

7. The valve of claim 6 in which said tubular loop is of essentially square shape.

8. The valve of claim 7 in which said tubular branches connect to the tubular loop at corners of said square shape, the direction of connection of at least some of said tubular branches at opposite corners extending transversely of the loop in a first direction, and the direction of connection of the remaining branches extending transversely of the loop in a direction opposite to said first direction.

9. The valve of claim 8 in which each tubular branch connects at a transverse angle with said tubular loop.

10. The valve of claim 6, in combination with a one-piece slide clamp capable of collapsing two opposed portions of said tubular loop, to provide selective flow control through said flow reversal valve depending on the position of said slide clamp.

11. The valve of claim 6 in which said flow reversal valve comprises part of an extracorporeal blood handling set.

12. The valve of claim 6 in which each tubular branch connects at a transverse angle with said tubular loop.

13. The valve of claim 6 in which said tubular loop occupies a plane.

14. The valve of claim 6 in which said tubular loop comprises a plurality of said tube sections connected together in rectangular form defining four corners, said tubular branches connecting to the loop at said corners.

15. The valve of claim 1, in combination with a one-piece slide clamp capable of collapsing two opposed portions of said tubular loop to provide selective flow control through said flow reversal valve depending on the position of said slide clamp.

16. The flow reversal valve of claim 1 which comprises part of an extracorporeal blood handling set.

17. The valve of claim 1 in which said tubular loop comprises a plurality of said tube sections connected together in rectangular form defining four corners, said tubular branches connecting to the loop at said corners.

18. A flow reversal valve for a tubular fluid flow set comprising at least four tubular branches and a tubular loop, said tubular loop having separate connections with each of said tubular branches at ends of said tubular branches, said tubular loop having clampable tube sections between said connections with said branches, said tubular loop and tubular branches defining a self-supporting structure that defines three dimensionally arranged flow paths through said tubular loop and tubular branches, to facilitate the directions in which said tubular branches extend for efficient operation.

19. The valve of claim 18 in which said tubular loop is of essentially square shape.

20. The valve of claim 18 in which said tubular loop comprises a plurality of said tube sections connected together in rectangular form defining four corners, said tubular branches connecting to the loop at said corners.

21. The valve of claim 20 in which a first pair of tubular branches extending from diagonally opposed corners of said loop extend in a first direction adjacent to said loop, and the remaining diagonally opposed, tubular branches extend parallel in the direction opposite to said first pair.
